(19) United States
(12) Patent Application Publication
(10) Pub. No.: US 2025/0188450 A1
(43) Pub. Date: Jun. 12, 2025

(54) UNIVERSAL SUPPRESSOR TRNAS AND USES THEREOF

(71) Applicant: hC Bioscience, Inc., Boston, MA (US)

(72) Inventor: David H. Altreuter, Wayland, MA (US)

(21) Appl. No.: 18/782,060

(22) Filed: Jul. 24, 2024

Related U.S. Application Data
(63) Continuation of application No. PCT/US2023/061249, filed on Jan. 25, 2023.
(60) Provisional application No. 63/302,885, filed on Jan. 25, 2022.

(51) Int. Cl. C12N 15/11; C12N 15/85

(52) U.S. Cl. CPC C12N 15/11; C12N 15/85; C12N 2310/531

(57) ABSTRACT

The present invention is related at least in part to tRNA therapeutic compositions that are specific to more than one stop codon.

### RELATED APPLICATIONS

This application claims the benefit under 35 U.S.C. § 119 (e) to U.S. Provisional Application No. 63/302,885, entitled “UNIVERSAL SUPPRESSOR tRNAS”, filed on Jan. 25, 2022, the entire contents of which are incorporated herein by reference.

### REFERENCE TO AN ELECTRONIC SEQUENCE LISTING

The contents of the electronic sequence listing (H101570007WO00-SEQ-ZJG.xml; Size: 19,216 bytes; and Date of Creation: Jan. 24, 2023) is herein incorporated by reference in its entirety.

### BACKGROUND

DNA molecules carry genetic information in the form of the sequence of the nucleotide bases that make up the DNA polymer. Only four nucleotide bases are utilized in DNA: adenine, guanine, cytosine, and thymine. This information, in the form of codons of three contiguous bases is transcribed into messenger RNA (mRNA), and then translated by transfer RNA (tRNA) and ribosomes to form proteins. Four nucleotide bases are utilized in RNA: adenine, guanine, cytosine, and uracil. The genetic code is the relation between a triplet codon and a particular amino acid. Sixty-four possible codon triplets form the genetic code, where three stop (also called “terminating” or “nonsense”) codons provide a signal to the translation machinery (cellular ribosomes) to stop protein production at the particular codon. The other sixty-one codon triplets (also called “sense codons”) correspond to one of the 20 standard amino acids.

### SUMMARY OF THE INVENTION

Premature termination codons (PTCs) caused by mutations such as nonsense mutations can result in a premature stop of protein translation, and therefore, may cause protein dysfunction and/or disease. Provided herein are approaches to provide tRNAs that can restore, at least partially, protein function. Such tRNAs can reverse or treat disease or disorders that are caused by or associated with one or more PTCs.

The invention is related, at least in part, to tRNA-mediated therapeutics that target nonsense mutations wherein the substituted amino acid may or may not be the native amino acid that was or would have been encoded (e.g., amino acid of the wild type protein) without the nonsense mutation. That is, the tRNA-mediated therapeutics can provide for “read through” of PTCs and/or at least partial, if not full, functional restoration of a gene product. Thus, the resulting gene product may or may not have amino acid sequence identity as the original, wild type, or non-disease-related gene product. It is proposed that tRNA compositions that target more than one possible premature termination codon and substitutes with an amino acid, such as serine, is a preferred embodiment of the invention. In one embodiment of any one of the compositions or methods provided herein, the tRNA composition targets all three possible stop codons and substitutes with an amino acid, such as serine.

While there are only 3 stop codons, there are 23 different nucleotide substitutions that can lead to a stop codon. For example, the UGA stop codon is one nucleotide distant from codons for arginine (CGA, AGA), glycine (GGA), leucine (UUA), serine (UCA), cysteine (UGU, UGC) and tryptophan (UGG). The most frequent nonsense mutations are CGA (arginine)->UGA and CAG (glutamine)->UGA. A disease state can result from the truncation of any protein induced by a premature termination codon. A disease-free or disease-reduced state can be induced by reading through the PTC with a suppressor tRNA that inserts an amino acid at the position erroneously coded by the PTC.

Whereas, specific rare genetic diseases resulting from a PTC may have a known specific nonsense mutation and therefore a specific restorative treatment suppressor tRNA, there are other disease states, including cancer, for which the specific PTC, or set of PTCs, may not be known. In these instances, the treatment of PTC(s) with suppressor tRNAs providing read through or functionally-analogous amino acid coding may be substantially therapeutic. It is noted that serine, as a hydrophilic amino acid of modest size, is likely in many cases to provide similar structural value as arginine, leucine, glutamine, cysteine or glycine, for example. It is further noted that certain aminoacyl tRNA synthetases do not substantially engage with the anticodon region of a tRNA while others do. Alanine, serine, and leucine tRNAs are recognized by their synthetases primarily through non-anticodon elements of the tRNA.

Thus, the use of tRNA compositions where at least two if not all three termination codons are targeted to substitute an amino acid, such as serine, can be used to treat diseases without the need to know the specific PTC(s) that give rise to or contribute to a disease.

Some aspects of the present disclosure provide compositions comprising more than one tRNA each with an anticodon specific for a stop codon, wherein the composition can target at least two stop codons, and wherein the tRNAs each can provide an amino acid to substitute in place of the stop codon to which it is specific.

In some embodiments, the composition comprises more than two tRNAs each with an anticodon specific for a stop codon, and wherein the composition can target all three stop codons. In some embodiments, the tRNAs each can provide the same amino acid to substitute in place of the stop codon to which it is specific. In some embodiments, the amino acid to substitute in place of the stop codons is serine. In some embodiments, the amino acid to substitute in place of the stop codons is arginine. In some embodiments, the amino acid to substitute in place of the stop codons is glutamine. In some embodiments, the amino acid to substitute in place of the stop codons is glutamic acid.

In some embodiments, the tRNAs can provide two different amino acids to substitute in place of the at least two stop codons to which they are specific. In some embodiments, the two amino acids to substitute in place of the stop codons are selected from arginine, glutamine, and glutamic acid. In some embodiments, the two amino acids to substitute in place of the stop codons are arginine and glutamine.

In some embodiments, the tRNAs can provide three different amino acids to substitute in place of the three stop codons to which they are specific. In some embodiments, the amino acids to substitute in place of the stop codons are arginine, glutamine, and glutamic acid.

In some embodiments, the stop codons are selected from TGA, TAA, and TAG.

In some embodiments, the composition further comprises a pharmaceutically acceptable carrier.

Further provided herein are oligonucleotides that encode the tRNAs described herein. In some embodiments, the oligonucleotide comprises the nucleotide sequence of any one of SEQ ID NOs: 1-14. A set of such oligonucleotides that together encode the tRNAs described herein are also provided. In some embodiments, each oligonucleotide in the set comprises a nucleotide sequence of any one of SEQ ID NOs: 1-14. In some embodiments, the oligonucleotide(s) are DNA. In some embodiments, the oligonucleotide(s) are RNA.

Further provided herein are expression cassettes comprising a promoter and a nucleic acid encoding the tRNAs, the oligonucleotide, or the set of oligonucleotides described herein. In some embodiments, the promoter is operably linked to a such nucleic acid, oligonucleotide, or set of oligonucleotides. In some embodiments, the set of expression cassettes together encode all of the tRNAs described herein. In some embodiments, one or more of the expression cassettes further comprises a terminator. In some embodiments, each expression cassette further comprises a terminator.

In some aspects, vectors comprising the oligonucleotide(s) or the expression cassette(s) described herein are provided. In some embodiments, the vector is a viral or plasmid vector.

Further provided herein are compositions comprising, the tRNAs, the oligonucleotide(s), the expression cassette(s), or the vectors described herein, and a pharmaceutically acceptable carrier. In some embodiments, the composition is comprised in a particle, such as a nanoparticle. In some embodiments, the particle is a liposome or a lipid nanoparticle. In some embodiments, the composition is comprised in an exosome.

Further provided herein are cells comprising the tRNAs, the oligonucleotide(s), the expression cassette(s), the vectors, or the compositions described herein.

Other aspects of the present disclosure provide methods of reading through one or more stop codons, comprising delivering the tRNAs, the oligonucleotide(s), the expression cassette(s), the vectors, or the compositions described herein to cells in an amount effective to read through the one or more stop codons.

Other aspects of the present disclosure provide methods of restoring at least partial protein function, comprising delivering the tRNAs, the oligonucleotide(s), the expression cassette(s), the vectors, or the compositions described herein to cells in an amount effective to restore at least partial protein function.

Other aspects of the present disclosure provide methods of reducing or inhibiting cell survival or proliferation, comprising delivering the tRNAs, the oligonucleotide(s), the expression cassette(s), the vectors, or the compositions described herein to cells in an amount effective to reduce or inhibit cell survival or proliferation.

In some embodiments, the cells are in vitro. In some embodiments, the cells are in vivo.

Other aspects of the present disclosure provide methods of treating a subject, comprising delivering the tRNAs, the oligonucleotide(s), the expression cassette(s), the vectors, or the compositions described herein to cells in an amount effective to treat the subject. In some embodiments, the subject has a disease caused by or attributed to one or more nonsense mutations or one or more premature termination codons. In some embodiments, the subject has a genetic disease or disorder. In some embodiments, the subject has a hyperproliferative disease or disorder. In some embodiments, the hyperproliferative disease or disorder is cancer. In some embodiments, the administration is systemic or local administration. In some embodiments, the subject is a human.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used in this disclosure is for the purpose of description and should not be regarded as limiting. The use of “including,” “comprising,” or “having,” “containing,” “involving,” and variations of thereof in this disclosure, is meant to encompass the items listed thereafter and equivalents thereof as well as additional items. As used in this specification and the appended claims, the singular forms “a,” “an” and “the” include plural referents unless the content clearly dictates otherwise.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

### BRIEF DESCRIPTION OF THE DRAWINGS

FIG. 1 provides a table of the genetic code.

FIG. 2 shows the general four-arm structure of tRNAs comprising a T-arm, a D-arm, an anticodon arm, and an acceptor stem (or arm) (SEQ ID NO: 15). These regions may also be referred to as ‘loops’ throughout.

FIG. 3 provides a map of a plasmid encoding one single tRNA.

FIG. 4 provides a map of a plasmid encoding two tRNAs.

FIG. 5 provides a map of a plasmid encoding three tRNAs.

FIG. 6 provides a map of a plasmid encoding three tRNAs (anti-codon edited (ACE) or native), and additionally encoding a transfection reporter (superfolder GFP).

FIG. 7 provides a graph showing that plasmids encoding universal suppressor tRNAs transfected into HEK293T PTC NLuc cells induced expression of nano-luciferase, while a control plasmid (hCR000.p014) lacking suppressor tRNA coding sequence did not.

FIG. 8 provides a Western Blot gel image showing increased expression of full-length p53 in HT1299 cancer cells containing an integrated CMV-promoted p53 gene having a premature TGA stop codon, which leads to a truncated p53 protein without the universal suppressor tRNA. A control plasmid lacking suppressor tRNA coding sequence (hCR000.p014) did not induce increased expression of full-length p53.

FIGS. 9A and 9B provide graphs showing the ratio of full-length p53 expression level (FIG. 9A) or truncated p53 expression level (FIG. 9B) in HT1299 cancer cells containing an integrated CMV-promoted p53 gene having a premature TGA stop codon and treated with plasmids encoding universal tRNAs, relative to the respective expression levels in untreated cells after normalizing on total protein. Cells treated with plasmid encoding universal suppressor tRNAs showed enhanced expression of full length p53 and reduced expression of truncated p53, while cells treated with a control plasmid lacking suppressor tRNA coding sequence (hCR000.p014) showed comparable levels of full-length p53 and truncated p53 as untreated cells.

### DETAILED DESCRIPTION

Provided herein is a therapeutic for treatment, such as for the treatment of cancer, wherein the therapeutic provides for more than one nonsense suppressor tRNA that are specific for more than one if not all stop codons. These nonsense suppressor tRNAs can provide for one or more amino acids, such as serine, arginine, glutamine, and/or glutamic acid, and read through the PTC(s). The nonsense suppressor tRNAs may not provide full and/or exact restoration of the native or wild type protein but can still have therapeutic effect even with partially restored or analogous function. In some embodiments, the tRNAs can target PTC(s) and substitute with serine, which can be an effective substitution in many cases due to the nature of the amino acid as well as synthetase recognition. In some embodiments, the tRNAs can target PTC(s) and substitute with arginine. In some embodiments, the tRNAs can target PTC(s) and substitute with glutamine. In some embodiments, the tRNAs can target PTC(s) and substitute with glutamic acid. In some embodiments, the tRNAs can target PTC(s) and substitute with more than one (e.g., two, three, or more) amino acids. In some embodiments, the more than one (e.g., two, three, or more) amino acids are selected from serine, arginine, glutamine, and glutamic acid.

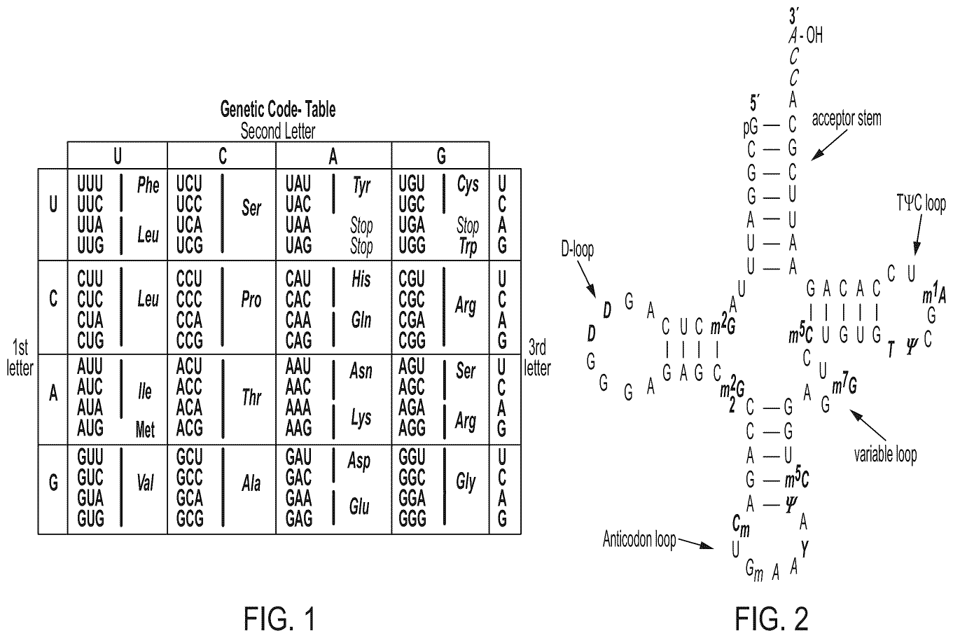

Transfer RNAs (tRNAs) are decoders of DNA and RNA “blueprints.” DNA transcription results in messenger RNAs (mRNAs) that encode primary amino acid structures that may be modified post-transcriptionally and that, upon interaction with ribosomes and tRNAs, eventually become folded or unfolded proteins. Once an mRNA engages with a ribosome, tRNAs deliver amino acids to the ribosome and form a chain of amino acids based on the code of the mRNA (FIG. 1).

tRNAs have a general four-arm structure comprising a T-arm, a D-arm, an anticodon arm, and an acceptor stem or arm (FIG. 2). The T-arm is made up of a “T-stem” and a “TY′C loop.” Any one of the tRNAs provided herein can comprise this four-arm structure. The tRNAs are approximately 100 nucleotides in length, in some embodiments, and can be readily introduced into cells.

The RNA molecules can be modified or engineered such that they can enable the systematic “recoding” of the genetic code. It has been found that a tRNA can be changed through molecular editing of the anticodon sequence within the tRNA. This approach allows for reprogramming an unwanted stop codon to be substituted with an amino acid.

Provided herein are compositions and methods related to a technology for the introduction of one or more suppressor tRNAs to diseased cells or tissues that can read through one or more PTCs. Particularly, the one or more suppressor tRNAs can broadly act on various premature termination codons (PTCs). The tRNA compositions provided herein can be engineered to target any one or more unwanted stop codons, such as any one or more of the stop codons provided herein. In some embodiments, the one or more amino acids that can be substituted is/are any amino acid(s) that is/are suitable for the present disclosure. Without wishing to be bound by any theory, serine, as a hydrophilic amino acid of modest size is expected to provide similar structural value as other amino acids, such as arginine, leucine, glutamine, cysteine or glycine. Accordingly, serine is expected to be an effective amino acid substitute in many cases for common PTCs. In some embodiments, the one or more tRNAs can be used to suppress erroneously coded PTC(s) and replace it with an amino acid (e.g., serine, arginine, glutamine, or glutamic acid) during polypeptide or protein production. In some embodiments, the one or more tRNAs can be used to suppress more than one type of erroneously coded PTC(s) and replace them with all with the amino acid (e.g., serine, arginine, glutamine, or glutamic acid), or with different amino acids (e.g., amino acids selected from serine, arginine, glutamine, or glutamic acid, or combinations thereof) during polypeptide or protein production.

As used herein, “premature termination codon” or PTC, refers to single nucleotide mutations that convert a canonical triplet nucleotide codon into one of three stop codons, e.g., TAG, TGA, or TAA. While there are only 3 stop codons currently known in the art, there are 23 different nucleotide substitutions that can lead to a stop codon. For example, the UGA stop codon can be derived from codons for arginine (CGA, AGA), glycine (GGA), leucine (UUA), serine (UCA), cysteine (UGU, UGC) and tryptophan (UGG). The presence of the stop codon, or a termination codon, signals a halt to protein synthesis (i.e., the termination of the translation process of the current protein). PTCs can cause truncation of a protein, thereby causing abnormalities and diseases.

In some embodiments, the present disclosure provides a composition comprising one or more tRNAs as described herein. In some embodiments, a composition described herein comprises more than one tRNA each with an anticodon specific for a stop codon, wherein the composition can target at least two stop codons (e.g., 2 or 3 stop codons selected from TAA, TGA, and TAG), and wherein the tRNAs each can provide an amino acid (e.g., a same amino acid or multiple different amino acids) to substitute in place of the stop codon to which it is specific.

In some embodiments, the composition comprises more than two tRNAs each with an anticodon specific for a stop codon, and wherein the composition can target all three stop codons (i.e., all of TAA, TGA, and TAG).

In some embodiments, a composition described herein comprises more than one tRNAs, wherein the tRNAs each can provide the same amino acid to substitute in place of the stop codon to which it is specific. In some embodiments, the amino acid to substitute in place of the stop codons is serine. In some embodiments, the amino acid to substitute in place of the stop codons is arginine. In some embodiments, the amino acid to substitute in place of the stop codons is glutamine. In some embodiments, the amino acid to substitute in place of the stop codons is glutamic acid.

In some embodiments, a composition described herein comprises more than one tRNAs, wherein the tRNAs can provide two different amino acids to substitute in place of the at least two stop codons to which they are specific. In some embodiments, the two amino acids to substitute in place of the stop codons are selected from arginine, glutamine, and glutamic acid. In some embodiments, the two amino acids to substitute in place of the stop codons are arginine and glutamine.

In some embodiments, a composition described herein comprises more than one tRNAs, wherein the tRNAs can provide three different amino acids to substitute in place of the three stop codons to which they are specific. In some embodiments, the amino acids to substitute in place of the stop codons are arginine, glutamine, and glutamic acid.

Further provided herein are oligonucleotides, or a set of oligonucleotides, encoding any one or more of the tRNAs as provided herein. In some embodiments, the oligonucleotide comprises a nucleotide sequence as set forth in any one of SEQ ID NOs: 1-14. In some embodiments, a composition described herein comprises oligonucleotides comprising nucleotide sequences selected from SEQ ID NOs: 1-14 or the tRNAs encoded by the oligonucleotides.

In some embodiments, the present disclosure provides a composition comprising one or more nucleic acids or expression constructs that encode the one or more tRNAs as described herein. In some embodiments, the compositions further comprise a pharmaceutically acceptable carrier. In some embodiments, the pharmaceutically acceptable carrier can be any carrier that is known in the art and is suitable for the present disclosure. In some embodiments, the present disclosure provides a cell that comprises one or more tRNAs or the one or more nucleic acids they encode as described herein. In some embodiments, the cell is a mammalian cell. In some embodiments, the cell is a human cell.

The present disclosure provides methods of treating or achieving read through of one or more PTCs by using one or more suppressor tRNAs which can provide functionally-analogous amino acid coding (e.g., serine, arginine, glutamine, or glutamic acid). The insertion of the functionally-analogous amino acid into the amino acid codon that is erroneously encoded by the PTC(s) can restore partial function, if not full, of gene products. In some embodiments, the restored gene product(s) may or may not be identical to the wild-type or the gene product(s) without the PTC(s).

The present disclosure provides methods of treating a subject with a disease or disorder. In some embodiments, the methods comprise administering to a subject one or more tRNAs or a composition as disclosed herein. In some embodiments, the disease or disorder may include a genetic disease or disorder or a hyperproliferative disease or disorder. In some embodiments, the disease or disorder may be cancer. In some embodiments, the disease or disorder may be any disease or disorder that can benefit from a tRNA-based treatment as provided herein.

In some embodiments, the protein expression or function is restored at least partially compared with a control. As used herein, an example of a “control” can be a tRNA that is not a suppressor tRNA as provided herein or that is not expected to read through a PTC. As another example, a control may be the same tRNA but without being able to substitute an amino acid of interest, such as a serine, into the position erroneously coded by the PTC(s). Comparisons of the improvement of the disease or disorder, the restoration or any other features can be assessed in vitro as known by the ordinarily skilled artisan or in vivo with the use of a test subject.

In some embodiments, the disease or disorder is improved (e.g., less severe) in the subject administered the tRNA(s) or composition provided herein by at least 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79%, 80%, 81%, 82%, 83%, 84%, 85%, 86%, 87%, 88%, 89%, 90%, 91%, 92%, 93%, 94%, 95%, 96%, 97%, 98%, or 99%, including all values in between, when compared with a control, such as a control tRNA.

As used herein, a “genetic disease or disorder” is a disease or disorder that is caused by mutations that are inherited or that arise within a subject's genetic code or that predisposes a subject to a disease or disorder. Any one of the compositions or methods provided herein can be for treating or preventing a genetic disease or disorder.

As used herein, “hyperproliferative disease or disorder” refers to any disease or disorder where there is an abnormally high rate of proliferation of cells by rapid division, substantial overproliferation, etc. Certain embodiments of the present disclosure provide a method of treating a disease or disorder, such as a hyperproliferative disease or disorder in a subject, such as a mammal. In certain embodiments, the mammal is human. Certain embodiments of the present disclosure provide a use of one or more tRNA(s) or composition as described herein to prepare a medicament useful for treating a disease or disorder, such as a hyperproliferative disease or disorder, in a subject, such as a mammal, such as a human. In certain embodiments, the therapy has potential use for the treatment/management of a disease or disorder, such as a hyperproliferative disease or disorder, including tumors, cancers, and neoplastic tissue, along with non-neoplastic or non-malignant hyperproliferative disorders. In certain embodiments, the hyperproliferative disease or disorder is cancer.

The tRNAs and nucleotide sequences encoding the tRNAs can be generated synthetically. Also, nucleotide sequences encoding several hundred human tRNAs are known and generally available to those of skill in the art through sources such as GenBank. In one embodiment of any one of the compositions or methods provided herein the tRNA(s) are selected from those provided in PCT/US2018/059065, the disclosure of the tRNAs and sequences of which are incorporated herein by reference in its entirety. In one embodiment of any one of the compositions or methods provided herein, the tRNA(s) are selected from those provided in PCT/US2018/059065 that can substitute with a serine. The structure of tRNAs is generally highly conserved, and tRNAs can be functional across species. Thus, bacterial or other eukaryotic tRNA sequences are also potential sources for the tRNAs provided herein. The determination of whether a particular tRNA or combination of tRNAs is functional as desired, such as in a desired mammalian cell, can be ascertained as described herein or through other experimentation that will be apparent to one of ordinary skill in the art with the benefit of the teachings provided herein.

The tRNA(s) may be in any form suitable, such as a recombinant plasmid that comprises a heterologous nucleic acid sequence, to be delivered to a target cell or subject, either in vitro or in vivo. The heterologous nucleic acid sequence encodes a gene product (e.g., a tRNA) of interest for the purposes of, for example, any one of the uses provided herein including disease treatment, and may, optionally, be in the form of an expression cassette. The term “recombinant” refers to a polynucleotide which either does not occur in nature or is linked to another polynucleotide in an arrangement not found in nature. The term “heterologous,” as used herein refers to a nucleic acid sequence obtained or derived from a genetically distinct entity from the rest of the entity to which it is being compared.

The present disclosure provides methods for delivering one or more tRNA(s) or composition provided herein to a subject or to cells of a subject. The tRNA(s) or composition as described herein can be delivered to a cell in vivo or in vitro. Administration to the cell can be accomplished by any means, including simply contacting the cell. The contact with the cells can be for any desired length of time. The cells can include any desired cell in humans as well as other large (non-rodent) mammals, such as primates, horse, sheep, goat, pig, and dog. Any one of the subjects provided herein can be a human or other mammal. The term “mammal” includes, but is not limited to, humans, mice, rats, guinea pigs, monkeys, dogs, cats, horses, cows, pigs, and sheep.

In some embodiments, administration can be systemic. In some embodiments, administration can be local. In some embodiments, administration can be direct delivery to the selected organ, oral, inhalation, intraocular, intravenous including facial vein injection and retroorbital injection, intracerebroventricular (ICV), intracisterna magna (ICM) injection, intramuscular, intrathecal, intracranial, subcutaneous, intradermal, intratumoral, and other parental routes of administration. Routes of administration may be combined, if desired. In some embodiments, the tRNA as disclosed herein can be administered via any route that is appropriate for the present disclosure.

Suitable methods for the delivery and introduction into a subject are also provided or otherwise understood in the art. In one embodiment, pharmaceutical compositions will comprise sufficient genetic material to produce a therapeutically effective amount of the tRNA(s) of interest. The tRNAs or genetic material that encodes the tRNA(s) can be delivered in an effective amount, and into a cell, such as with endogenous tRNA synthetase. A tRNA synthetase is considered to be “endogenous” to a cell if it is present in the cell into which a tRNA is introduced according to the present invention. As will be the apparent to those of ordinary skill in the art, a tRNA synthetase may be considered to be endogenous for these purposes whether it is naturally found in cells of the relevant type, or whether the particular cell at issue has been engineered or otherwise manipulated by the hand of man to contain or express it.

In some embodiments, the tRNA can be formulated in a pharmaceutical composition. The pharmaceutical compositions may also contain a pharmaceutically acceptable excipient. Such excipients include any pharmaceutical agent that may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

As is apparent to those skilled in the art in view of the teachings of this specification, an effective amount of the tRNAs provided may be empirically determined. Administration can be effected in one dose, continuously or intermittently, throughout the course of treatment. Methods of determining the most effective means and dosages of administration may vary with the composition of the therapy, target cells, and the subject being treated, etc. Single and multiple administrations can be carried out with the dose level and pattern being selected by the treating physician.

Vehicles including water, aqueous saline, artificial CSF, or other known substances can be employed with the subject invention. To prepare a formulation, the purified composition can be isolated. The composition may then be adjusted to an appropriate concentration and packaged for use.

As used herein, the terms “treat” and “treatment” refer to both therapeutic treatment and measures that can alleviate symptoms or provide some benefit to a subject, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition, disease or disorder.

The phrase “therapeutically effective amount” means an amount of one or more compounds or a composition of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

The terms “cancer” and “cancerous” refer to or describe the physiological condition in mammals that is typically characterized by unregulated cell growth. A “tumor” comprises one or more cancerous cells. Examples of cancer include, but are not limited to, carcinoma, malignancies, etc.

The tRNA(s) or composition described herein may be administered so as to result in a reduction in at least one symptom associated with a disease or disorder, such as a genetic disease or disorder or a hyperproliferative disease or disorder (e.g., cancer). The amount administered will vary depending on various factors including, but not limited to, the composition chosen, the particular disease, the weight, the physical condition, and the age of the mammal. Such factors can be readily determined by the clinician employing animal models or other test systems that are known to the art.

Administration of the tRNA(s) or composition in accordance with the present invention may be continuous or intermittent, depending, for example, upon the recipient's physiological condition, whether the purpose of the administration is therapeutic, and other factors known to skilled practitioners. The administration may be essentially continuous over a preselected period of time or may be in a series of spaced doses.

One or more suitable unit dosage forms having one or more tRNA(s) or composition of the invention may be formulated and can be administered by a variety of routes. When the agents of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. Pharmaceutical formulations can be prepared by procedures known in the art using well-known and readily available ingredients. The agents of the invention can also be formulated as solutions appropriate for administration. The pharmaceutical formulations of the agents of the invention can also take the form of an aqueous or anhydrous solution or dispersion, or alternatively the form of an emulsion or suspension.

Thus, the agent(s) may be formulated for administration and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in a suitable vehicle, e.g., sterile, pyrogen-free water, before use. It will be appreciated that the unit content of active ingredient or ingredients contained in an individual dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0 and water.

Any of the compositions provided herein can be placed in contact with, administered to or introduced into a cell with genetic transfer methods, such as transfection. Thus, any of the compositions provided herein can be included with or in a gene delivery vehicle. The gene delivery vehicle can be any delivery vehicle known in the art and can include naked nucleic acid that is facilitated by a receptor and/or lipid mediated transfection.

The compositions provided herein can be contacted with cells or delivered or administered to a subject within a particle, such as a nanoparticle. A particle, such as a nanoparticle, can be, but is not limited to, lipid-based nanoparticles (also referred to herein as lipid nanoparticles, i.e., nanoparticles where the majority of the material that makes up their structure are lipids) and/or particles with a combination of nanomaterials. The particles may be a variety of different shapes, including but not limited to spheroidal, cuboidal, pyramidal, oblong, cylindrical, toroidal, and the like.

In some embodiments, particles, such as nanoparticles, may comprise one or more lipids. In some embodiments, particles, such as nanoparticles, may comprise liposomes. In some embodiments, particles, such as nanoparticles, may comprise a lipid bilayer. In some embodiments, particles, such as nanoparticles, may comprise a lipid monolayer. In some embodiments, particles, such as nanoparticles, may comprise a micelle.

The tRNAs or compositions provided herein can be contacted with cells or delivered or administered to a subject with an extracellular vesicle or exosome. Generally, exosomes are nano-sized extracellular vesicles (EVs) (30-150 nm in diameter) which can be formed and released by many mammalian cells. The EVs or exosomes can be loaded with agent(s) of interest, for example by pre-treatment of cells with the agent and then isolation of loaded EVs or exosomes.

EVs or exosomes can be derived from human embryonic kidney cells, bone marrow stem cells, immature dendritic cells, red blood cells as well as from milk. EVs or exosomes can be isolated and purified with a number of different techniques. Such methods include, but are not limited to ultracentrifugation, ultrafiltration, size exclusion chromatography (SEC), precipitation with polymers, and separation by affinity-based methods, such as immunomagnetic-based isolation.

Electroporation may also be used.

In an embodiment of any one of the compositions or methods provided herein, a nucleic acid sequence encoding a tRNA is in a closed-end form, such as in a plasmid, nanoplasmid or minicircle. In an embodiment of any one of the compositions or methods provided herein, the nucleic acid sequence encoding a tRNA is in the form of a minicircle or microthread.

The term “minicircle”, as used herein, refers to small circular DNA fragments that are largely or completely free of non-essential prokaryotic elements. Minicircles include circular forms of DNA without prokaryotic elements and/or in which prokaryotic elements have been removed. Minicircles can be from a parental plasmid where bacterial DNA sequences have been excised. The minicircle may be in the form of any suitable recombinant plasmid that comprises a heterologous nucleic acid sequence to be delivered to a target cell, either in vitro or in vivo. The preparation of minicircles have been described in the art (e.g., in Nehlsen et al., Gene Ther. Mol. Biol. 10:233-244, 2006; and Kay et al., Nature Biotechnology. 28:1287-1289, 2010). The preparation can, for example, follow a two-step procedure: (i) production of a ‘parental plasmid’ (bacterial plasmid with eukaryotic inserts); and (ii) induction of a site-specific recombinase at the end of this process. These steps can be followed by the excision of prokaryotic vector parts via recombinase-target sequences and recovery by capillary gel electrophoresis.

As a nonlimiting example, a minicircle may be produced as follows. An expression cassette, which comprises the polynucleotide coding sequence along with regulatory elements for its expression, is flanked by attachment sites for a recombinase. A sequence encoding the recombinase is located outside of the expression cassette and includes elements for inducible expression (such as, for example, an inducible promoter). Upon induction of recombinase expression, the vector DNA is recombined, resulting in two distinct circular DNA molecules. One of the circular DNA molecules is relatively small, forming a minicircle that comprises the expression cassette for the polynucleotide; this minicircle DNA vector is devoid of any bacterial DNA sequences. The second circular DNA sequence contains the remaining vector sequence, including the bacterial sequences and the sequence encoding the recombinase. The minicircle DNA containing the polynucleotide sequence can then be separately isolated and purified. In some embodiments, a minicircle DNA vector may be produced using plasmids similar to pBAD.ϕ.C31.hFIX and pBAD.ϕ.C31.RHB. See, e.g., Chen et al. (2003) Mol. Ther. 8:495-500, or as otherwise provided herein.

Examples of recombinases that may be used for creating a minicircle include, but are not limited to, Streptomyces bacteriophage ϕ31 integrase, Cre recombinase, and the λ integrase/DNA topoisomerase IV complex. Each of these recombinases catalyzes recombination between distinct sites. For example, ϕ31 integrase catalyzes recombination between corresponding attP and attB sites, Cre recombinase catalyzes recombination between loxP sites, and the λ integrase/DNA topoisomerase IV complex catalyzes recombination between bacteriophage λ attP and attB sites.

Published U.S. application No. 20170342424 also describes a system making use of a parent plasmid which is exposed to an enzyme which causes recombination at recombination sites, thereby forming a (i) minicircle including the polynucleotide sequence and (ii) miniplasmid comprising the remainder of the parent plasmid. One recombination site is modified at the 5′ end such that its reaction with the enzyme is less efficient than the wild type site, and the other recombination site is modified at the 3′ end such that its reaction with the enzyme is less efficient than the wild type site, and the other recombination site is modified at the 3′ end such that its reaction with the enzyme is less efficient than the wild type site, both modified sites being located in the minicircle after recombination.

Removal of prokaryotic sequences ideally should be efficient, using the smallest possible excision site, while creating supercoiled DNA minicircles which consist solely of gene expression elements under appropriate—preferably mammalian—control regions. Some techniques for minicircle production use bacterial phage lambda (λ) integrase mediated recombination to produce minicircle DNA. See, for example, Darquet, et al. 1997 Gene Ther 4 (12): 1341-9; Darquet et al. 1999 Gene Ther 6 (2): 209-18; and Kreiss, et al. 1998 Appl Micbiol Biotechnol 49 (5): 560-7).

Kits for producing minicircle DNA are known in the art and are commercially available (System Biosciences, Inc., Palo Alto, Calif.). For example, a MC-Easy™ (Cat #MN920A-1, SBI System Biosciences) Minicircle DNA production kit can be used to obtain high-quality minicircle DNA. Information on minicircle DNA is provided in Dietz et al., Vector Engineering and Delivery Molecular Therapy (2013); 21 8, 1526-1535 and Hou et al., Molecular Therapy—Methods & Clinical Development, Article number: 14062 (2015) doi: 10.1038/mtm.2014.62. More information on Minicircles is provided in Chen Z Y, He C Y, Ehrhardt A, Kay M A. Mol Ther. 2003 September; 8 (3): 495-500 and Minicircle DNA vectors achieve sustained expression reflected by active chromatin and transcriptional level. Gracey Maniar L E, Maniar J M, Chen Z Y, Lu J, Fire A Z, Kay M A. Mol Ther. 2013 January; 21 (1): 131.

In some embodiments, the closed-end form is a supercoiled helix. DNA supercoiling refers to the amount of twist in a particular DNA strand. Supercoiled DNA can be positively supercoiled DNA or negatively supercoiled DNA. As used herein, “supercoiled DNA” refers to a DNA molecule, or fragment of a DNA molecule, wherein one or both DNA strands comprise increased twisting compared to the amount of twisting in a reference state known as “relaxed B-form” DNA. In a “relaxed” double-helical segment of DNA, the two strands twist around the helical axis once every 10.4-10.5 base pairs of sequence. A given DNA strand may be “positively supercoiled” or “negatively supercoiled” (i.e., more or less tightly wound). Supercoiling creates twist strain in the DNA strand. The amount of a strand's supercoiling affects a number of biological processes, such as compacting DNA and regulating access to the genetic code (which strongly affects DNA metabolism and possibly gene expression). Certain enzymes (e.g., topoisomerases) are capable of increasing or decreasing the amount of twisting (e.g., supercoiling) in a DNA strand in order to facilitate functions such as DNA replication and transcription. If a DNA segment under twist strain is closed into a circle by joining its two ends, and then allowed to move freely, it may form a supercoiled structure. Examples of supercoiled structures of circular DNA molecules include, but are not limited to a figure-eight structure, a plectonemic structure, or a toroidal structure.

In an embodiment of any one of the compositions or methods provided herein, an oligonucleotide described herein further comprises a promoter. Such an oligonucleotide may be comprised in a vector. The promoter characteristically has a specific nucleotide sequence necessary to initiate transcription. In some embodiments, an oligonucleotide as provided herein comprises a nucleic acid sequence capable of directing expression of a particular nucleotide sequence in an appropriate cell, which may include a promoter operably linked to the nucleotide sequence of interest that may also be operably linked to termination signals (or terminator). An oligonucleotide as provided herein may be in a recombinant form useful for heterologous expression. In certain embodiments, the promoter is a regulatable promoter. In certain embodiments, the promoter is a constitutive promoter. The promoter to drive expression of the sequence encoding tRNA(s) to be delivered can be any desired promoter, selected by known considerations, such as the level of expression of a nucleic acid functionally linked to the promoter and the cell type in which the vector is to be used. Promoters can be an exogenous or an endogenous promoter.

In an embodiment of any one of the compositions or methods provided herein, promoters may be between (35-105 bp in size). The promoters may be any known promoters, including native tRNA leader sequences, which sequences may be ˜50-60 bp in size. In an embodiment of any one of the compositions or methods provided herein, the promoter may be reduced-sequence or re-configured promoters. Example promoter sequences that may be comprise in any one of the oligonucleotides or vectors provided herein include, but are not limited to, any one of the sequences provided herein or otherwise known in the art.

Optionally, the oligonucleotides or vectors provided herein may further include additional sequences (e.g., 3′ tRNA tail or trailer sequences). Such sequences may be between 2-20 bp in length. They may include natural sequences or engineered variants with 3-10 consecutive “T” residues.

The present disclosure also provides a cell containing a tRNA(s), oligonucleotide(s), vector, or other composition of the invention described herein. The cell may be mammalian, such as human. According to one aspect, a cell expression system is provided. The expression system comprises a cell and an oligonucleotide, or set thereof, or vector as provided herein. Expression cassettes include, but are not limited to, plasmids, viral vectors, and other vehicles for delivering heterologous genetic material to cells. The cell expression system can be formed in vivo.

In some embodiments of any one of the tRNAs or other compositions of the invention provided herein, the tRNA(s) or composition(s) is one not found in nature.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1. Design and Generation of Universal Suppressors

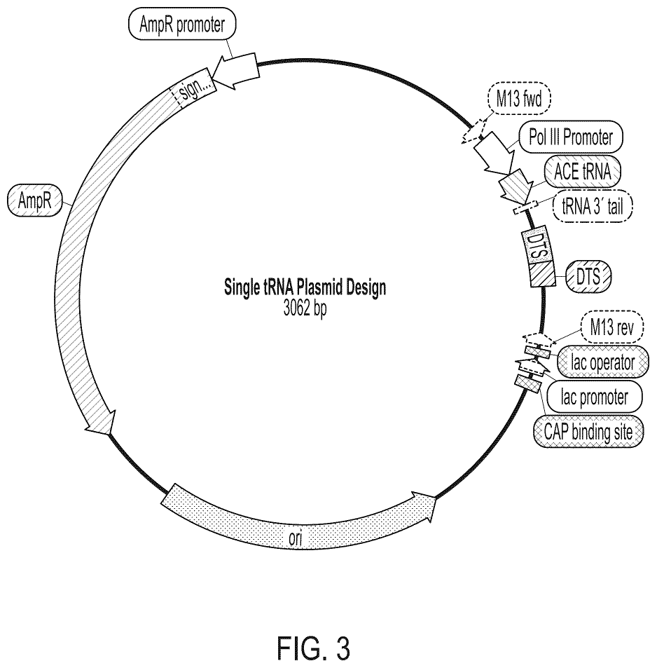

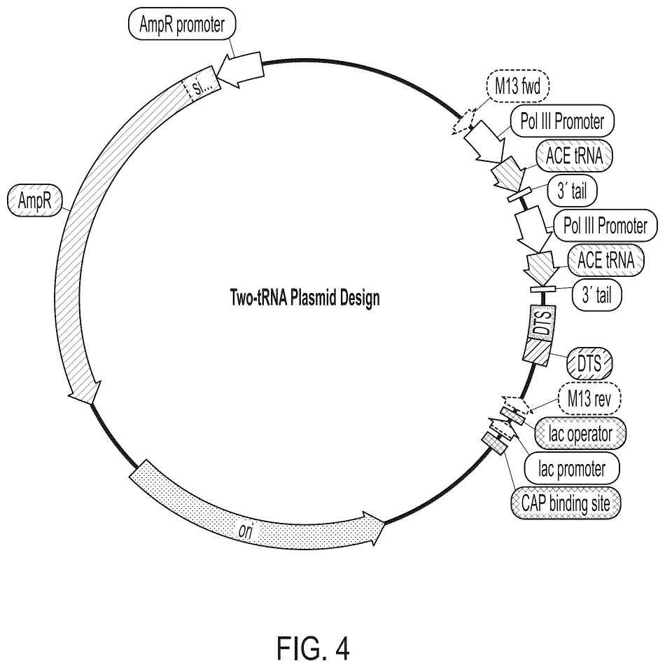

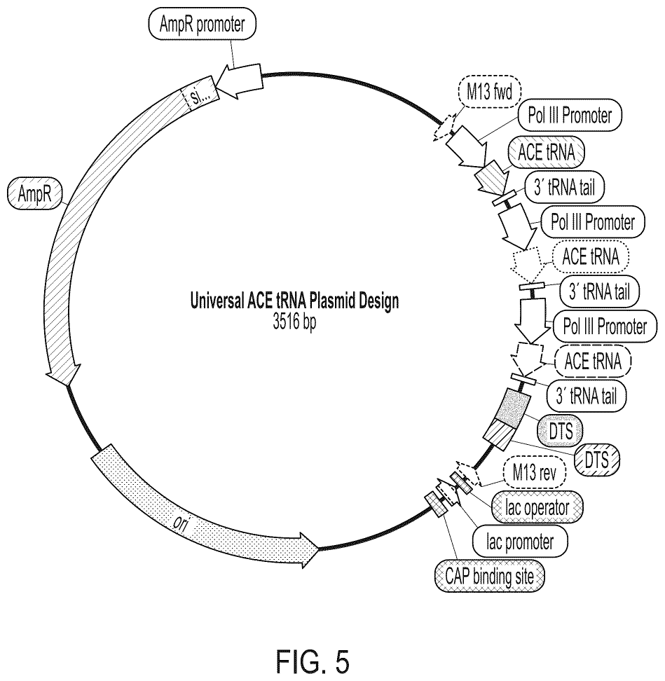

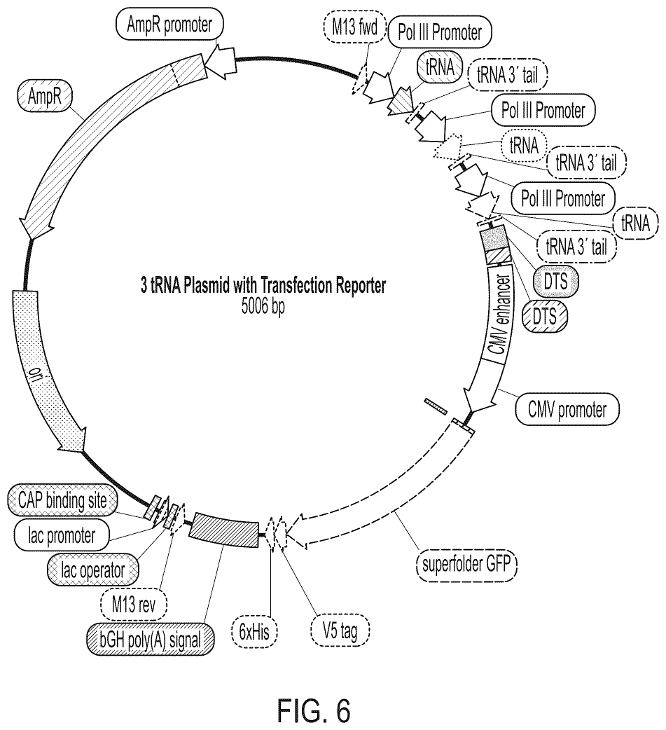

Anti-codon edited (ACE) tRNA suppressor sequences were designed by replacing the anticodons of exemplary native human tRNA sequences with engineered anticodons matched to TGA, TAG, and TAA stop codons. The DNA sequences for these ACE tRNA were synthesized and incorporated into plasmid constructs as described in FIGS. 3-6. FIG. 3 provides the design for plasmids bearing a single tRNA sequence, FIG. 4 for plasmids bearing two tRNA sequences, FIG. 5 for plasmids bearing three tRNA sequences, and FIG. 6 for plasmids bearing three tRNA sequences and also a transfection reporter sequence. All plasmids were based on a pUC57 backbone, utilize the same DNA polymerase III promoter, have the same tRNA trailer/termination sequence, and contain the same DTS sequences. Plasmid size varies slightly within any given design depending on the length of the encoded tRNA sequence. Table 1 provides a listing of the ACE tRNAs uniquely encoded by the synthesized plasmids.

TABLE 1
Synthesized Universal Suppressor plasmids and associated control plasmids

| Name of tNRA | | ACE tRNA Sequence Origin | | | Transfection |
|---|---|---|---|---|---|
| Composition | Classification | TGA | TAG | TAA | Reporter |
| hCR004.p001 | Universal\hat{ } Serine | Ser-TGA-2-1 | Ser-TGA-2-1 | Ser-TGA-2-1 | — |
| hCR004.p002 | Universal Serine | Ser-TGA-3-1 | Ser-TGA-3-1 | Ser-TGA-3-1 | — |
| hCR004.p003 | Universal Serine | hC-Ser001 | hC-Ser001 | hC-Ser001 | |
| hCR001.p001 | ACE TGA-only | Arg-CCG-2-1 | — | — | — |
| hCR002.p001 | ACE TAG-only | — | Gln-CTG-3-1 | — | — |
| hCR002.p002 | ACE TAA-only | — | — | Gln-TTG-1-1 | — |
| hCR003.p001 | ACE TAA-only | — | — | Glu-TTC-2-1 | — |
| hCR201.p001 | ACE (TGA + TAG) | Arg-CCG-2-1 | Gln-CTG-3-1 | — | — |
| hCR201.p002 | ACE (TGA + TAA) | Arg-CCG-2-1 | — | Gln-TTG-1-1 | — |
| hCR201.p003 | ACE (TAG + TAA) | — | Gln-CTG-3-1 | Gln-TTG-1-1 | — |
| hCR201.p005 | ACE (TAG + TAA) | | Gln-CTG-3-1 | Glu-TTC-2-1 | — |
| hCR301.p001 | Universal Multi\hat{ }\hat{ } | Arg-CCG-2-1 | Gln-CTG-3-1 | Gln-TTG-1-1 | — |
| hCR301.p002 | Universal Multi | Arg-CCG-2-1 | Gln-CTG-3-1 | Glu-TTC-2-1 | — |
| hCR301.p003 | Universal Multi | Arg-TCT-1-1 | Gln-CTG-3-1 | Gln-TTG-1-1 | wt sfGFP |
| hCR000.p022 | NATIVE Control | Arg-TCT-1-1* | Gln-CTG-3-1* | Gln-TTG-1-1* | wt sfGFP |
| hCR004.p004 | Universal Serine | Ser-TGA-2-1 | Ser-TGA-2-1 | Ser-TGA-2-1 | wt sfGFP |
| hCR000.p023 | NATIVE Control | Ser-TGA-2-1* | Ser-TGA-2-1* | Ser-TGA-2-1* | wt sfGFP |
| hCR000.p014 | No-tRNA Control** | — | — | — | — |

*Synthesized as the native, non-ACE, to serve as control sequences to the ACE-tRNA-bearing plasmids.
**No-tRNA Control plasmid hCR000.p014 is a pUC backbone plasmid wherein the pol III promoter, tRNA, tRNA tail, and DTS are absent and replaced with a randomized non-coding sequence.
\hat{ }tRNA compositions classified with “universal” means the composition contains a mixture of suppressor tRNAs that can target all three stop codons TAG, TGA, and TAA.
\hat{ }\hat{ }tRNA compositions classified with “multi” means the composition contains a mixture of suppressor tRNAs that can provide at least two different amino acid at different stop codons

TABLE 2
tRNA sequences corresponding to Table 1

| tRNA | PTC Target | tRNA Encoding Sequence* |
|---|---|---|
| Ser-TGA-2-1 | TGA | GTAGTCGTGGCCGAGTGGTtAAGGCGATGGACTtcaA |
| | | ATCCATTGGGGTTTCCCCGCGCAGGTTCGAATCCTG |
| | | CCGACTACG (SEQ ID NO: 1) |
| Ser-TGA-2-1 | TAG | GTAGTCGTGGCCGAGTGGTtAAGGCGATGGACTctaA |
| | | ATCCATTGGGGTTTCCCCGCGCAGGTTCGAATCCTG |
| | | CCGACTACG (SEQ ID NO: 2) |
| Ser-TGA-2-1 | TAA | GTAGTCGTGGCCGAGTGGTtAAGGCGATGGACTttaA |
| | | ATCCATTGGGGTTTCCCCGCGCAGGTTCGAATCCTG |
| | | CCGACTACG (SEQ ID NO: 3) |
| Ser-TGA-3-1 | TGA | GTAGTCGTGGCCGAGTGGTLAAGGCGATGGACTtcaA |
| | | ATCCATTGGGGTCTCCCCGCGCAGGTTCGAATCCT |
| | | GCCGACTACG (SEQ ID NO: 4) |
| Ser-TGA-3-1 | TAG | GTAGTCGTGGCCGAGTGGTLAAGGCGATGGACTctaA |
| | | ATCCATTGGGGTCTCCCCGCGCAGGTTCGAATCCT |
| | | GCCGACTACG (SEQ ID NO: 5) |
| Ser-TGA-3-1 | TAA | GTAGTCGTGGCCGAGTGGTtAAGGCGATGGACTttaA |
| | | ATCCATTGGGGTCTCCCCGCGCAGGTTCGAATCCT |
| | | GCCGACTACG (SEQ ID NO: 6) |
| hC-Ser001 | TGA | GTAGTGGTGGCCGAGTGGTTAAGGCGATGGACTtca |
| | | AATCCATTGGGGTCTCCCCGCGCAGGTTCGAATCC |
| | | TGCCCACTACG (SEQ ID NO: 7) |
| hC-Ser001 | TAG | GTAGTGGTGGCCGAGTGGTTAAGGCGATGGACTcta |
| | | AATCCATTGGGGTCTCCCCGCGCAGGTTCGAATCC |
| | | TGCCCACTACG (SEQ ID NO: 8) |
| hC-Ser001 | TAA | GTAGTGGTGGCCGAGTGGTTAAGGCGATGGACTtta |
| | | AATCCATTGGGGTCTCCCCGCGCAGGTTCGAATCC |
| | | TGCCCACTACG (SEQ ID NO: 9) |
| Arg-CCG-2-1 | TGA | GACCCAGTGGCCTAATGGAtAAGGCATCAGCCTtcaG |
| | | AGCTGGGGAtTGTGGGTTCGAGTCCCATCTGGGTCG |
| | | (SEQ ID NO: 10) |
| Gln-CTG-3-1 | TAG | GGTTCCATGGTGTAATGGTgAGCACTCTGGACTctaA |
| | | ATCCAGCGaTCCGAGTTCGAGTCTCGGTGGAACCT |
| | | (SEQ ID NO: 11) |
| Gln-TTG-1-1 | TAA | GGTCCCATGGTGTAATGGTtAGCACTCTGGACTttaA |
| | | ATCCAGCGaTCCGAGTTCAAATCTCGGTGGGACCT |
| | | (SEQ ID NO: 12) |
| Glu-TTC-2-1 | TAA | TCCCACATGGTCTAGCGGTtAGGATTCCTGGTTttaAC |
| | | CCAGGCGGCCCGGGTTCGACTCCCGGTGTGGGAA |
| | | (SEQ ID NO: 13) |
| Arg-TCT-1-1 | TGA | GGCTCCGTGGCGCAATGGATAGCGCATTGGACTtca |
| | | AATTCAAAGGTTCCGGGTTCGAGTCCCGGCGGAGT |
| | | CG (SEQ ID NO: 14) |

*The sequences in Table 2 are DNA sequences encoding the indicated tRNAs. Actual tRNAs have Us where Ts are present in the coding sequences provided Table 2.

### Example 2. Rescue of a TGA Premature Termination Codon Using Universal Suppressors in a HEK293T PTC-Reporter Cell Line

HEK293T cells were transfected using piggyBac plasmids and antibiotic selection to yield a stable cell line with an integrated CMV-promoted Nanoluciferase (NLuc) gene having a TGA stop codon at N160. Cells were trypsinized and counted for cell concentration using DeNovix Cell Drop instrument. Cells were seeded into 96-well plates 10,000 cells/well in 100 μL of medium without penstrep. Lipofectamine 3000 was used to transfect the cells as per the manufacturer's instructions at the concentrations listed in Table 3. Lipofectamine reagent was made up and incubated for 15 minutes prior to addition to cells. 10 μL of mastermix was added to each well.

TABLE 3
HEK293T PTC NLuc Cell Treatments

| | | | Test | Treatment |
|---|---|---|---|---|
| | | # wells per | Concentrations | Duration |
| Group | Treatment | Concentration | (ug/mL) | (h) |
| 1 | None (cells only) | 4 | 0, 0, 0 | 24 |
| 2 | hCR004.p001 | 4 | 0.1, 0.3, 1.0 | 24 |
| 3 | hCR004.p002 | 4 | 0.1, 0.3, 1.0 | 24 |
| 4 | hCR301.p001 | 4 | 0.1, 0.3, 1.0 | 24 |
| 5 | hCR301.p002 | 4 | 0.1, 0.3, 1.0 | 24 |
| 6 | hCR201.p003 | 4 | 0.1, 0.3, 1.0 | 24 |
| 7 | hCR201.p005 | 4 | 0.1, 0.3, 1.0 | 24 |
| 8 | hCR000.p014 | 4 | 0.1, 0.3, 1.0 | 24 |

For the nanoLuciferase Assay, nanoLuciferase reagent and substrate were equilibrated to room temperature. Cell plates were incubated at room temperature. 100 μL of reagent+substrate was added to each test well in the 96-well plate. Plates were shaken for 10 minutes prior to reading the signal on the plate reader.

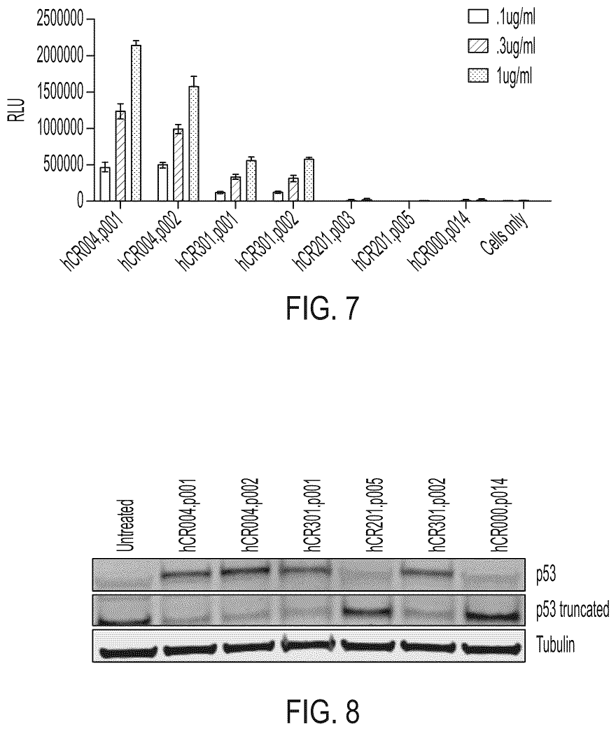

As shown in FIG. 7, the universal suppressor plasmid designs provided significant and dose dependent positive Nanoluciferase signal, indicating restorative readthrough of the TGA PTC. Strong signal was observed for the serine-based universal suppressor plasmids (hCR004.p001 and hCR004.p002) as well as the multi-tRNA universal suppressor designs that contained an ACE arginine tRNA suppressor of TGA (hCR301.p001 and hCR301.p002). No significant signal was observed for the control plasmid designs that lacked a TGA suppressor element (hCR201.p003, hCR201.p005 and hCR000.p014) or for untreated cells.

### Example 3. Recovery of p53 PTC in the HT1299 Cancer Cell Line Using Universal Suppressor Plasmids

H1299 cells were transfected using pCDNA-3.1 with puromycin antibiotic selection to yield a stable cell line with an integrated CMV-promoted p53 gene having a TGA stop codon at R213.

Lipofectamine 3000 was used to transfect the cells as per the manufacturer's instructions at the concentrations listed in Tables 4. Lipofectamine reagent was made up and incubated for 15 minutes prior to addition to cells. 200 μL of mastermix was added to each well.

TABLE 4
H1299 p53 PTC Design (6 wells)

| | | # | Concentration | Time |
|---|---|---|---|---|
| Group | Treatment | wells | (ug/mL) | (hrs) |
| 1 | None (Cells Only) | 1 | 0 | 48 |
| 2 | hCR004.p001 | 1 | 1 | 48 |
| 3 | hCR004.p002 | 1 | 1 | 48 |
| 4 | hCR301.p001 | 1 | 1 | 48 |
| 5 | hCR201.p005 | 1 | 1 | 48 |
| 6 | hCR301.p002 | 1 | 1 | 48 |
| 7 | hCR000.p014 | 1 | 1 | 48 |

After transfection, media was removed from cells. Wells were washed 3× with PBS. 100 μL of RIPA buffer and protease/phosphatase inhibitors were added to each well. Cells were scraped and collected in 1.7 mL Eppendorf tubes. Cells were lysed on ice for 1 hour and clarified at 15,000 rpm for 20 minutes. Protein concentrations were normalized via BCA. Western blots were run using ˜15-30 μg of protein in 10-well gels at 100V for 60 minutes. Gels were transferred to nitrocellulose membranes via iBlot 2.0 and blocked. Membranes were incubated with 1° antibody overnight and 2° antibody the following day for 1 hour. Membranes were washed 3× in TBST following incubations. Membranes were imaged using Licor.

The Western blot data (FIG. 8) shows that serine-based universal suppressors (hCR004.p001 and hCR004.p002) and Multi-tRNA universal suppressors containing an ACE arginine tRNA suppressor of TGA (hCR301.p001 and hCR301.p002) substantially recover full-length p53 protein and reduce the presence of PTC-truncated p53. The multi-ACE tRNA plasmid that lacked a TGA suppressor sequence (hCR201.p005) served as a negative control and, like the untreated cells, did not evidence significant full-length p53 protein.

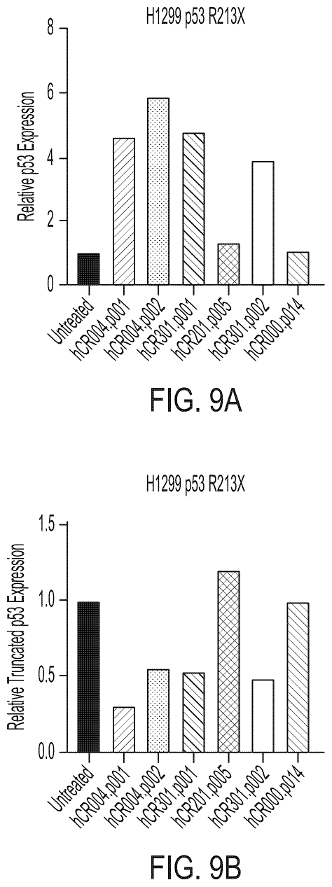

Based on the Western blot data, ratios of p53 recovery versus untreated cells and retention of truncated p53 versus untreated cells, were calculated. The results, shown in FIGS. 9A and 9B, respectively, confirm substantial full-length p53 expression in cells treated with the Universal Suppressor constructs and not in the negative control.

### EQUIVALENTS

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising more than one tRNA each with an anticodon specific for a stop codon, wherein the composition can target at least two stop codons, and wherein the tRNAs each can provide an amino acid to substitute in place of the stop codon to which it is specific.

2. The composition of claim 1, wherein the composition comprises more than two tRNAs each with an anticodon specific for a stop codon, and wherein the composition can target all three stop codons.

3. The composition of claim 1, wherein the tRNAs each can provide the same amino acid to substitute in place of the stop codon to which it is specific.

4. The composition of claim 1, wherein the amino acid to substitute in place of the stop codons is serine, arginine, glutamine, or glutamic acid.

5.-7. (canceled)

8. The composition of claim 1, wherein the tRNAs can provide two different amino acids to substitute in place of the at least two stop codons to which they are specific.

9. The composition of claim 8, wherein the two amino acids to substitute in place of the stop codons are selected from arginine, glutamine, and glutamic acid.

10. The composition of claim 8, wherein the two amino acids to substitute in place of the stop codons are arginine and glutamine.

11. The composition of claim 2, wherein the tRNAs can provide three different amino acids to substitute in place of the three stop codons to which they are specific.

12. The composition of claim 8, wherein the amino acids to substitute in place of the stop codons are arginine, glutamine, and glutamic acid.

13. The composition of claim 1, wherein the stop codons are selected from TGA, TAA, and TAG.

14. The composition of claim 1, wherein the composition further comprises a pharmaceutically acceptable carrier.

15. An oligonucleotide that encodes the tRNAs of claim 1.

16.-20. (canceled)

21. An expression cassette comprising a promoter and a nucleic acid encoding the tRNAs of claim 1.

22.-23. (canceled)

24. A vector comprising the oligonucleotide of claim 15.

25. (canceled)

26. A composition comprising, the tRNAs of claim 1, and a pharmaceutically acceptable carrier.

27.-29. (canceled)

30. A cell comprising the tRNAs of claim 1.

31. A method of reading through one or more stop codons, comprising delivering the tRNAs of claim 1 to cells in an amount effective to read through the one or more stop codons.

32. A method of restoring at least partial protein function, comprising delivering the tRNAs of claim 1 to cells in an amount effective to restore at least partial protein function.

33. A method of reducing or inhibiting cell survival or proliferation, comprising contacting cells with the tRNAs of claim 1 in an amount effective to reduce or inhibit cell survival or proliferation.

34.-35. (canceled)

36. A method of treating a subject, in need thereof, comprising administering the tRNAs of claim 1 to cells in an amount effective to treat the subject.

37.-41. (canceled)


SEQUENCE LISTING (15 sequences)

SEQ ID NO: 1 - DNA, 82 bp, synthetic construct
  source 1..82: mol_type = other DNA
gtagtcgtggccgagtggttaaggcgatggacttcaaatccattggggtttccccgcgcaggttcgaatcctgccgactacg

SEQ ID NO: 2 - DNA, 82 bp, synthetic construct
  source 1..82: mol_type = other DNA
gtagtcgtggccgagtggttaaggcgatggactctaaatccattggggtttccccgcgcaggttcgaatcctgccgactacg

SEQ ID NO: 3 - DNA, 82 bp, synthetic construct
  source 1..82: mol_type = other DNA
gtagtcgtggccgagtggttaaggcgatggactttaaatccattggggtttccccgcgcaggttcgaatcctgccgactacg

SEQ ID NO: 4 - DNA, 82 bp, synthetic construct
  source 1..82: mol_type = other DNA
gtagtcgtggccgagtggttaaggcgatggacttcaaatccattggggtctccccgcgcaggttcgaatcctgccgactacg

SEQ ID NO: 5 - DNA, 82 bp, synthetic construct
  source 1..82: mol_type = other DNA
gtagtcgtggccgagtggttaaggcgatggactctaaatccattggggtctccccgcgcaggttcgaatcctgccgactacg

SEQ ID NO: 6 - DNA, 82 bp, synthetic construct
  source 1..82: mol_type = other DNA
gtagtcgtggccgagtggttaaggcgatggactttaaatccattggggtctccccgcgcaggttcgaatcctgccgactacg

SEQ ID NO: 7 - DNA, 82 bp, synthetic construct
  source 1..82: mol_type = other DNA
gtagtggtggccgagtggttaaggcgatggacttcaaatccattggggtctccccgcgcaggttcgaatcctgcccactacg

SEQ ID NO: 8 - DNA, 82 bp, synthetic construct
  source 1..82: mol_type = other DNA
gtagtggtggccgagtggttaaggcgatggactctaaatccattggggtctccccgcgcaggttcgaatcctgcccactacg

SEQ ID NO: 9 - DNA, 82 bp, synthetic construct
  source 1..82: mol_type = other DNA
gtagtggtggccgagtggttaaggcgatggactttaaatccattggggtctccccgcgcaggttcgaatcctgcccactacg

SEQ ID NO: 10 - DNA, 73 bp, synthetic construct
  source 1..73: mol_type = other DNA
gacccagtggcctaatggataaggcatcagccttcagagctggggattgtgggttcgagtcccatctgggtcg

SEQ ID NO: 11 - DNA, 72 bp, synthetic construct
  source 1..72: mol_type = other DNA
ggttccatggtgtaatggtgagcactctggactctaaatccagcgatccgagttcgagtctcggtggaacct

SEQ ID NO: 12 - DNA, 72 bp, synthetic construct
  source 1..72: mol_type = other DNA
ggtcccatggtgtaatggttagcactctggactttaaatccagcgatccgagttcaaatctcggtgggacct

SEQ ID NO: 13 - DNA, 72 bp, synthetic construct
  source 1..72: mol_type = other DNA
tcccacatggtctagcggttaggattcctggttttaacccaggcggcccgggttcgactcccggtgtgggaa

SEQ ID NO: 14 - DNA, 73 bp, synthetic construct
  source 1..73: mol_type = other DNA
ggctccgtggcgcaatggatagcgcattggacttcaaattcaaaggttccgggttcgagtcccggcggagtcg

SEQ ID NO: 15 - RNA, 76 nt, unidentified
  source 1..76: mol_type = tRNA
  modified_base 10: mod_base = m2g
  modified_base 26: mod_base = m22g
  modified_base 32: mod_base = cm
  modified_base 34: mod_base = gm
  modified_base 39: mod_base = p
  modified_base 40: mod_base = m5c
  modified_base 46: mod_base = m7g
  modified_base 49: mod_base = m5c
  modified_base 55: mod_base = p
  modified_base 58: mod_base = m1a
  modified_base 54: mod_base = OTHER; note = Thymine
gcggatttagctcagddgggagagcgccagactgaayanctggaggtcctgtgtncgatccacagaattcgcacca